(19) United States
(12) Patent Application Publication
(10) Pub. No.: US 2026/0224466 A1
(43) Pub. Date: Aug. 6, 2026

(54) UV BLOCKING COMPOSITIONS AND METHODS

(71) Applicant: Trustees of Trufts College, Medford, MA (US)

(72) Inventors: Fiorenzo G. Omenetto, Lexington, MA (US); Giulia Guidetti, Somerville, MA (US); Erika Bechtold, Newton, MA (US); Gregory Scott Lynam, Sunset, SC (US)

(21) Appl. No.: 19/324,640

(22) Filed: Sep. 10, 2025

Related U.S. Application Data
(63) Continuation of application No. PCT/US2024/019738, filed on Mar. 13, 2024.
(60) Provisional application No. 63/489,969, filed on Mar. 13, 2023.

(51) Int. Cl. A61K 8/73; A61K 8/02; A61K 8/9789; A61Q 17/04

(52) U.S. Cl. CPC A61K 8/731; A61K 8/0241; A61K 8/9789; A61Q 17/04; A61K 2800/412

(57) ABSTRACT

A method of preparing a UV and/or visible light-blocking composition is disclosed. The method includes the generation of visually glittery microparticles from dried Lunaria septa and the use of this composition in skin care products, sunscreens, and other products is described.

This application claims benefit of and is a continuation of International Patent Application No. PCT/US2024/019738 (Attorney Docket. No. 2095.0598), filed Mar. 13, 2024, and entitled “UV BLOCKING COMPOSITIONS AND METHODS,” International Pub. No. WO 2024/215431, which is hereby incorporated by reference in its entirety for all purposes.

International Patent Application No. PCT/US2024/019738 claims the benefit of and priority to the following provisional application, which is hereby incorporated by reference in its entirety for all purposes: U.S. Patent Application Ser. No. 63/489,969 (Attorney Docket. No. 2095.0489), filed Mar. 13, 2023.

### STATEMENT REGARDING FEDERALLY SPONSORED RESEARCH

This invention was made with government support under grant numbers N00014-19-1-2399 and N00014-13-1-0596, both awarded by the US Navy, Office of Naval Research. The government has certain rights in the invention.

### REFERENCE TO A SEQUENCE LISTING

Not applicable.

### FIELD OF THE INVENTION

The disclosed technology is generally directed to a composition that is designed to interact with or attenuate light. More particularly the technology is directed to a novel composition including microparticles that reflect, scatter, and/or absorb light, such as UV, infrared, and/or visible light.

### BACKGROUND

Prolonged exposure to UV light (100 nm-400 nm) is largely dangerous for humans. Some wavelengths penetrate the atmosphere (UVA, 315-400 nm), while other wavelengths are partially blocked by the atmosphere (UVB, 280-315 nm) or absorbed by the ozone layer (UVC, 100 nm-280 nm). Exposure to the lower energy wavelengths can do harm. UV radiation is a proven human carcinogen, causing basal cell carcinoma (BCC), squamous cell carcinoma (SCC) and melanoma. Unprotected exposure to UVA and UVB damages the DNA in skin cells, producing genetic defects, or mutations, that can lead to skin cancer and premature aging. UV rays can also cause eye damage, including cataracts and eyelid cancers. These types of cancers often appear on sun-exposed areas of skin. Over the last 20 years, rates of skin cancer have dramatically increased, with rates of SCC increasing by as much as 300%.[1]

Currently available UV filtering materials such as those used in skin creams and sunscreens are either based on physical reflectors or chemical compounds that interact and/or absorb the energy from UV radiation. Physical reflectors such as titanium dioxide (TiO₂) and zinc oxide (ZnO) have a white chalky appearance when applied in a topical cream and are currently used as white pigments in various commercial applications despite the known harmful effects on the environment and on animals. These materials are non-sustainable as they produce peroxide when in contact with water, which has been reported to be detrimental to the environment and biodiversity (e.g., damaging phytoplankton, bleaching of coral reefs).[2] Chemical absorption compounds used in UV filtering materials are traditionally based on aromatic compounds (e.g., avobenzone, oxybenzone, and octocrylene). The safety of these compounds for human use has been questioned and furthermore they have been implicated as damaging to marine ecosystems and are thought to contribute to bleaching of coral reefs. Because of this, several chemical sunscreens are now banned in coastal island communities around the world (e.g. Hawaii).

Naturally-derived compounds which act as UV filters, such as lignin, have been investigated as UV blocking materials. Lignin is an aromatic polymer with phenolic groups that are UV-absorbing and provide free radical scavenging ability. As the second-most abundant biopolymer after cellulose, lignin is highly sustainable. However, a variety of chromophores responsible for a dark color are introduced into lignin during the isolation processes and under harsh conditions such as high temperature. Therefore, lignin currently has limited applications as a topical skin care cream or sunscreen due to the dark appearance of this family of materials caused by their isolation process.

UV blocking materials are needed that are effective, sustainable, environmentally friendly, and safe for human use.

### SUMMARY

In an aspect, a method of preparing a visible light-blocking and/or UV light-blocking composition is disclosed. The method comprises the step of submerging dried Lunaria septa in a thermal bath at a temperature of below −10° C., below −50° C., below −100° C., below −150° C., below −195° C. or in liquid nitrogen for at least 10 seconds, at least 30 seconds, at least 1 minute, at least 2 minutes, at least 2 minutes 30 seconds, or at least 3 minutes. The method further comprises the step of fragmenting dried Lunaria septa to microparticles while maintaining a temperature of the dried Lunaria septa below 15° C. The method further comprises the step of fractionating the microparticles.

In another aspect, a visually glittery composition is disclosed herein, wherein the visually glittery composition comprises fragmented and fractionated dried Lunaria septa, wherein the dried Lunaria septa are from a Lunaria plant harvested when the seedpods possess a broadband surface reflectance that is at least 40%, at least 50%, at least 60%, at least 70%, at least 80%, or at least 90% of a normalized maximum for each wavelength between 400 nm and 700 nm, wherein the reflectance is normalized to a white diffuser. In an aspect, the visually glittery composition is combined with a carrier.

In another aspect, a method of using cellulose microparticles for visible light protection, infrared light protection, and UV light protection is disclosed, where the method comprises the steps of producing cellulose microparticles from Lunaria septa, combining the particles with at least one carrier at or above 1 wt % particle concentration to generate a formulation, and applying this formulation to skin. In an aspect, the microparticles are between 10 μm and 100 μm in diameter and the microparticles show at least 50% of a normalized maximum for each wavelength between 400 nm and 700 nm, and wherein the reflectance is normalized to a white diffuser.

In another aspect, a visually glittery composition is disclosed. The visually glittery composition comprises a particle comprising lignin and cellulose. In an aspect, the particle comprises an array of parallel-arranged cellulose tubes, each tube having a cross-sectional diameter between 5 μm and 30 μm. In an aspect, the particle shows at least 50% of a normalized maximum for each wavelength between 400 nm and 700 nm, wherein the reflectance is normalized to a white diffuser. In an aspect, the particle possesses a broadband surface reflectance that is at least 40%, at least 50%, at least 60%, at least 70%, at least 80%, or at least 90% of a normalized maximum for each wavelength between 400 nm and 700 nm, wherein the reflectance is normalized to a white diffuser. In an aspect, the visually glittery composition comprises a carrier suitable for use on human skin. In an aspect, the carrier comprises at least one of the following components: a UVA filter, a UVB filter, an emollient, a moisturizer, or a thickening agent. In an aspect, wherein the particle imbues the composition with light-scattering effects.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A) Photograph of Lunaria annua seed pod showing the matte valve, seed, septum and funicle components. FIG. 1B) Photograph of septa from Lunaria rediviva.

FIG. 2A) Top view SEM image of a L. annua septum showing multidomain structure made by 1D arrays of cylindrical cells oriented in parallel fashion inside each domain. Scalebar is 100 μm. FIG. 2B) A schematic representation of the multidomain structure of the septum.

FIG. 3A) A cross-sectional SEM image of the septum shows the bilayer arrangement of the hollow cell arrays. Scalebar is 10 μm. FIG. 3B) A schematic representation with measurements taken from the SEM image.

FIG. 4A) A high-magnification cross-sectional SEM image of an individual hollow cell. Scalebar is 2 μm. FIG. 4B) The thin-film assembly in the cell wall cladding and alignment of the nanofibrils along the cell circumference. FIG. 4C) A schematic representation of a cross-section of a cell with aligned nanofibrils in the cell wall and a layered cell wall.

FIG. 5A) Bright-field reflection micrograph of the septum surface observed in top view showing thin-film interference-like colors spanning the entire visible spectrum. FIG. 5B) Reflectance spectra collected within single-colored regions, reflecting magenta (1), blue (2), and green (3), and corresponding optical micrographs (white circle indicates the fiber collection spot, Ø~4 μm, normalized to a white diffuser). Normalization with respect to a silver mirror is a standard reference and is typically used when evaluating strongly reflecting materials. Normalization with respect to a white diffuser is generally used when estimating the overall “whiteness” of a material. This type of reference is used for materials that are more scattering.

FIG. 6A) Bright-field reflection micrograph of the septum surface observed in top view showing thin-film interference-like colors spanning the entire visible spectrum. FIG. 6B) Average reflectance spectrum of the L. rediviva septa collected over large areas (collection spot Ø~60 μm, normalized to a silver mirror) as indicated by the solid black line and standard deviation by the shaded areas. N=30, reported values are average±standard deviation. Lunaria samples were collected from several sites for microscopic comparison, including FIG. 6C) Lunaria rediviva Slovenia Sample 10% 4×; FIG. 6D) Lunaria annua China Sample 10% 4×; FIG. 6E) Lunaria rediviva Slovenia Sample 10% 4×; FIG. 6F) Lunaria annua Tennessee Sample 10% 4×; FIG. 6G) Lunaria rediviva Slovenia Sample 33% 10×; FIG. 6H) Lunaria annua China Sample 33% 10×; FIG. 6I) Lunaria rediviva Slovenia Sample 33% 10×; and FIG. 6J) Lunaria annua Tennessee Sample 33% 10×. Optical magnification is represented by ‘x’. 4×=400% and 10×=1,000% optical magnification. The % represents digital magnification. A magnification of 10% 4× is 400% optical magnification plus 40% digital magnification. A magnification of 33% 10× is 1,000% optical magnification plus another 330% digital magnification.

FIG. 7A) Average reflectance spectrum of the Lunaria annua septa collected over large areas (collection spot Ø~60 μm, normalized to a white diffuser) as indicated by the solid black line and standard deviation by the shaded areas. FIG. 7B) Average reflectance spectrum of the Lunaria annua septa collected over large areas (collection spot Ø~60 μm, normalized to a silver mirror) as indicated by the solid black line and standard deviation by the shaded areas. FIG. 7C) Comparison of the average reflectance of the Lunaria annua (light gray band) and of the Lunaria rediviva (dark gray band) collected over large areas (collection spot Ø~60 μm, normalized to a silver mirror). For each graph: N=30, reported values are average±standard deviation.

FIG. 8. Photograph of Lunaria septa after grinding and sieving with a 700 μm sieve.

FIG. 9. After grinding, the Lunaria septa flakes are heterogeneous in size and shape. The bilayer structure of the Lunaria septum is retained as well as the hierarchical assembly of the fibrils as confirmed by optical and electron microscopy.

FIG. 10A depicts bright-field reflection micrographs of a flake and FIG. 10B depicts bright-field reflection micrographs of a fiber (FIG. 10B) μmicroparticle showing thin-film interference colors.

FIG. 11. Top view SEM images of a large (Left) and of a small (Right) μmicroparticle showing a similar morphology to the septa before any grinding.

FIG. 12. Bright-field reflection micrographs of the microparticles after size sorting through sieves with various mesh sizes.

FIG. 13A) FTIR absorbance spectra for the Lunaria septum (1) and valve (2) showing aromatic vibrations at ~1596 cm⁻¹ and 1506 cm⁻¹. FIG. 13B) Absorbance of the Lunaria septum (1) and of the Lunaria microparticles (2) compared to the absorbance of a commercially available dark lignin (3) and of the substrate, an empty well (4).

FIG. 14A) Average reflectance spectrum of the Lunaria whole (integer) septa, of the fine microparticles (53 μm fraction), and of the coarse microparticles (53-106 μm fraction) collected over large areas (collection spot Ø~60 μm, normalized to a silver mirror) as indicated by the solid black lines and standard deviation by the shaded areas. N=30, reported values are average±standard deviation. (B, C, and D) Bright-field reflection micrographs of the integer septa (FIG. 14B) and of the fine (FIG. 14C) and coarse (FIG. 14D) μmicroparticles.

FIG. 15. A 25 wt % water-based aloe cream was used to suspend the Lunaria flakes at 0.3 wt %, 0.6 wt %, or 1.2 wt %.

FIG. 16. (A-B) Absorbance of “LuMo Fine” cream series (FIG. 16A) and the “LuMo Coarse” cream series (FIG. 16B) at various loading compared to the absorbance of commercial moisturizing lotion (MO), of sunscreen cream (MOSPF), and of the substrate (Empty). (FIG. 16C-FIG. 16D) Corresponding absorbance evaluated at λ=300 nm (FIG. 16C) and at λ=300 nm (FIG. 16D). (FIG. 16E-FIG. 16F) Average reflectance spectra collected over large areas (collection spot Ø~60 μm, normalized to a silver mirror) for the Lunaria based creams made with fine (FIG. 16E) and coarse (FIG. 16F) microparticles compared to the commercial moisturizing lotion and to the sunscreen cream.

FIG. 17A) Macroscopic picture of volunteer arm on which swatches of the creams were applied. The black rectangle highlights the region where the creams were applied. (FIG. 17B) Macroscopic picture of the cream swatches just applied on the skin (FIG. 17B) and after drying (FIG. 17C): from top to bottom, swatch of the commercially available sunscreen cream (MOSPF30), the moisturizing cream (Mo), the fine Lunaria cream at 5 wt % (LuMo-Fine-5 wt %), and the coarse Lunaria cream at 5 wt % (LuMo-Coarse-5 wt %). (FIG. 17D) High magnification pictures of the dried fine (top) and (coarse) Lunaria creams once fully dried on the skin of the volunteer.

FIG. 18A depicts UV absorbance at 350 nm and FIG. 18B depicts UV absorbance at 400 nm for various concentrations of Lunaria annua diluted in either oil or primer.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to surface patterning are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

### Compositions

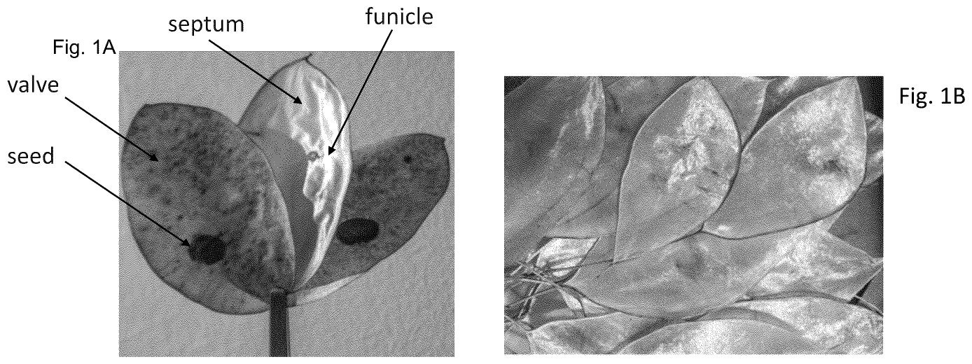

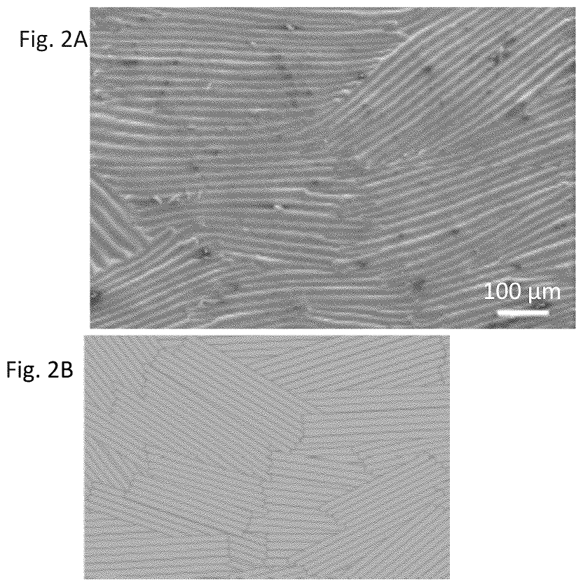

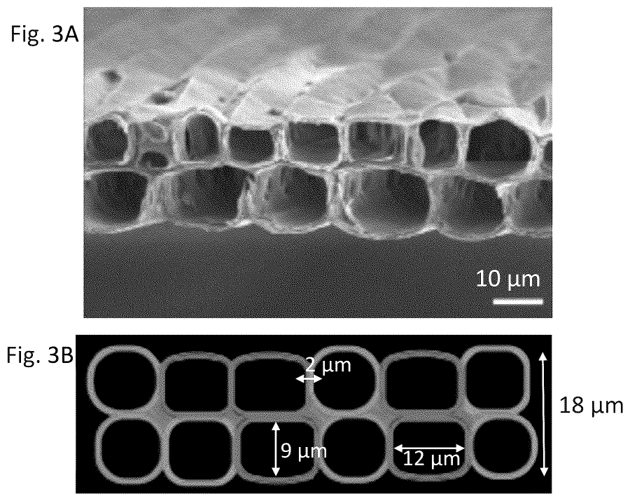

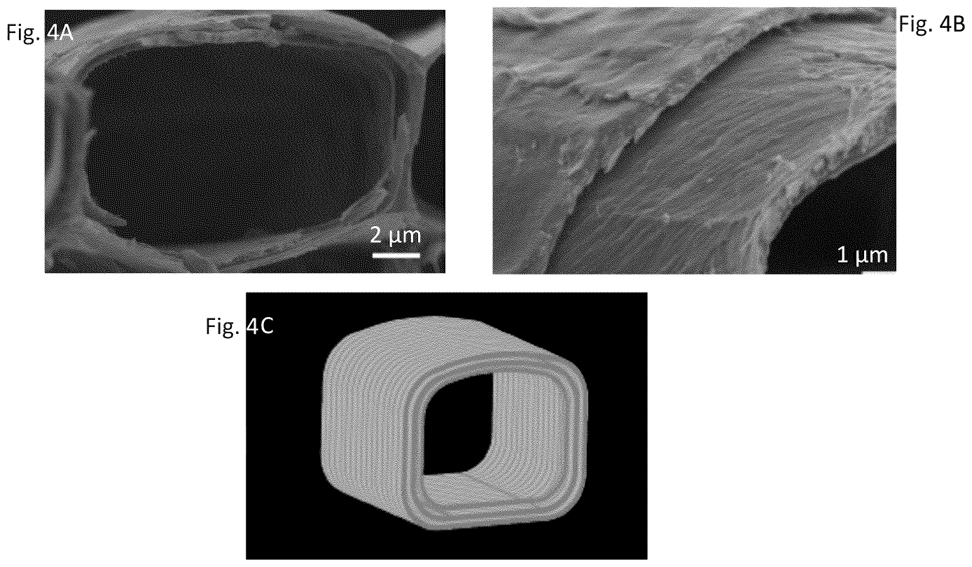

The present disclosure provides novel compositions including particles that provide a visibly glittery appearance and can be formulated into compositions that can be used as UV and/or visible light filters. According to an aspect disclosed herein, the compositions can include microparticles derived from Lunaria seedpod septa. The structure and reflective properties of Lunaria seedpod septa are described in “Silique valves as sails in anemochory of Lunaria (Brassicaceae)” C. Leins, P. Fligge, K. Erbar, Plant Biol. 2018, 20, 238 [3] and “Multiscale assembly of reflective cellulose sheets in Lunaria annua,” by G. Guidetti, H. Sun, B. Marelli, F. G. Omenetto, Sci. Adv. 2020, 6, eaba8966 [4], incorporated herein by reference in its entirety. Briefly, the seedpods from Lunaria (including for example L. rediviva and L. annua) contain cellulose and aromatic compounds such as lignin. The macroscopic and microscopic structural details of Lunaria seedpods are shown in FIGS. 1-7. The seedpods consist of a capsule having two external matte valves and an internal septum (FIG. 1A, Lunaria annua seedpod, and 1B, septa from Lunaria rediviva). Lunaria septa have a distinctive silvery-white reflective appearance at the macroscale that originates from microscale structure. The IR absorption properties of the septum (inner) and valve (outer) components of the Lunaria seedpod, which are disclosed in U.S. Ser. No. 63/489,969 and can be provided to an examiner upon request, confirms the presence of lignin. FIG. 2 shows the multidomain arrangement of cellulose fibers in the cells of the septum, where domains are groups of parallel fibers. These cellulose fibers induce thin-film interference-like colors at the microscale. A cross-sectional view of a septum in FIG. 3 shows the fibers are hollow, grouped in domains where the adjacent fibers are oriented in parallel and are further arranged in bilayer arrays. FIG. 4 shows the cell wall includes a multilayered thin-film assembly of parallel fibers.

As used herein, visually glittery is understood to describe the shimmery or glowing appearance of the inventive Lunaria particles and/or compositions disclosed herein. This appearance can be a desired trait for make-up and cosmetic products to add shimmer and/or impart light-reflecting and/or diffracting

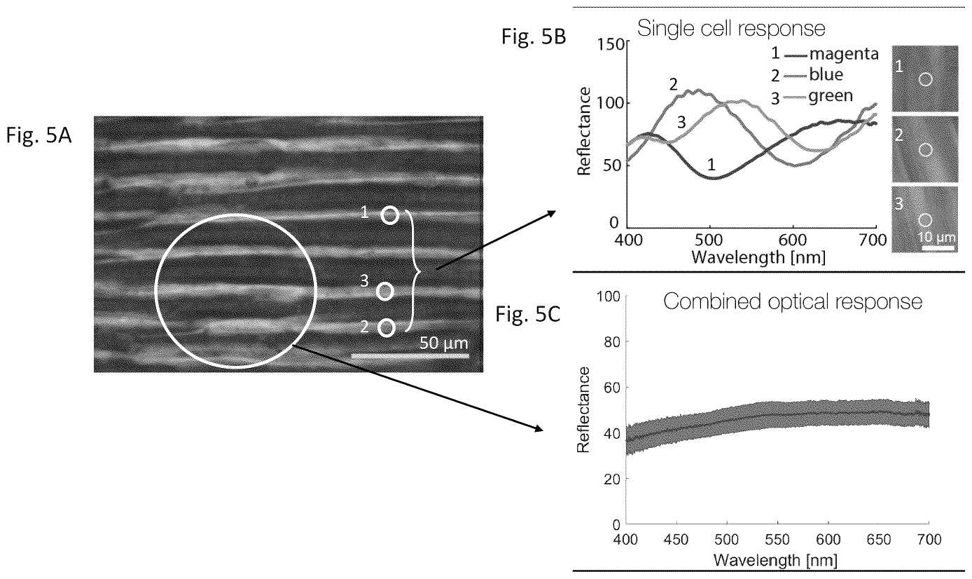

As shown in FIG. 5, individual cells can be individually observed to reflect different colors of light. This reflectance intensity modulation (FIG. 5B) is due to variations in the multilayered thin-film assembly, for example, differences in spacing between the multiple layers of fibers in the cell wall. Macroscopically, the reflected colors merge to create a broadband response and the silvery-white color of the septa.

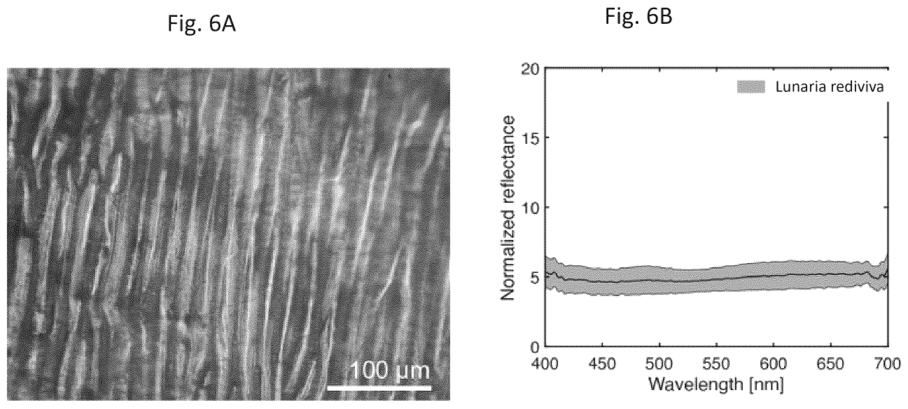

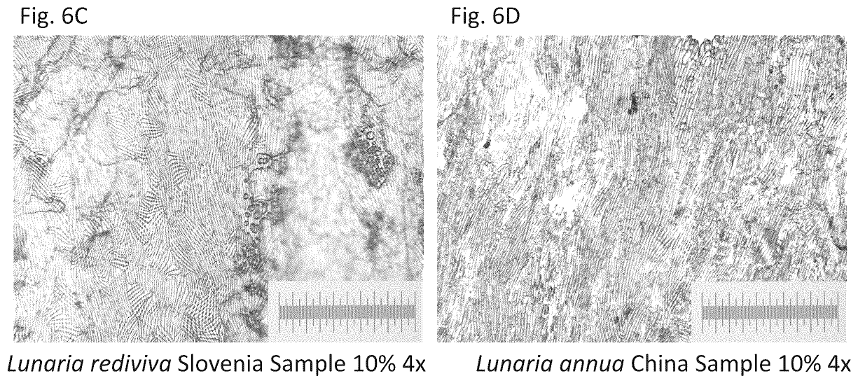

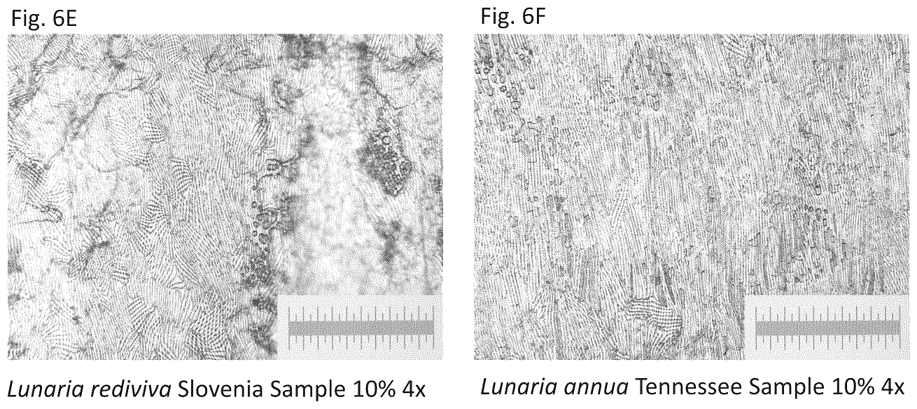

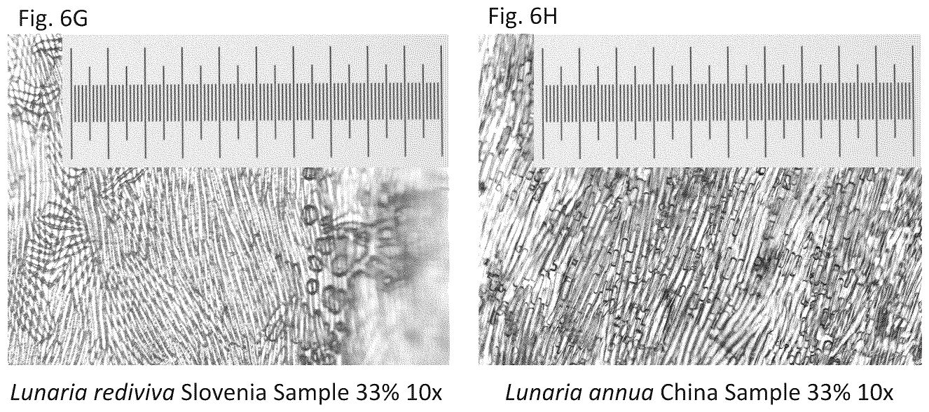

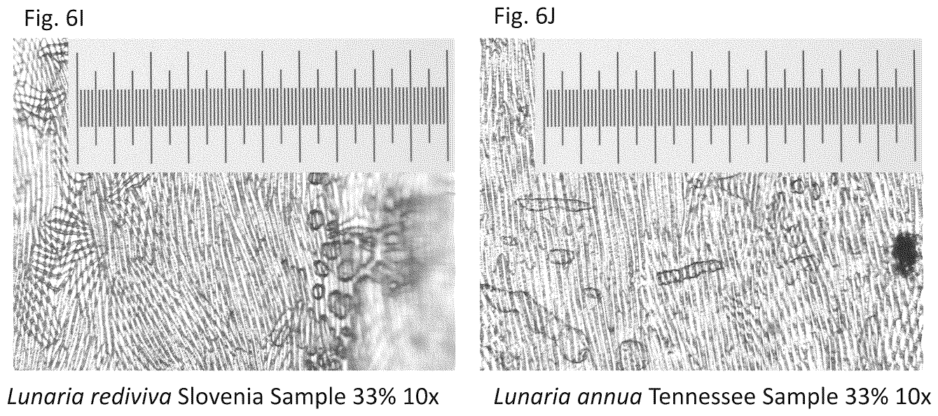

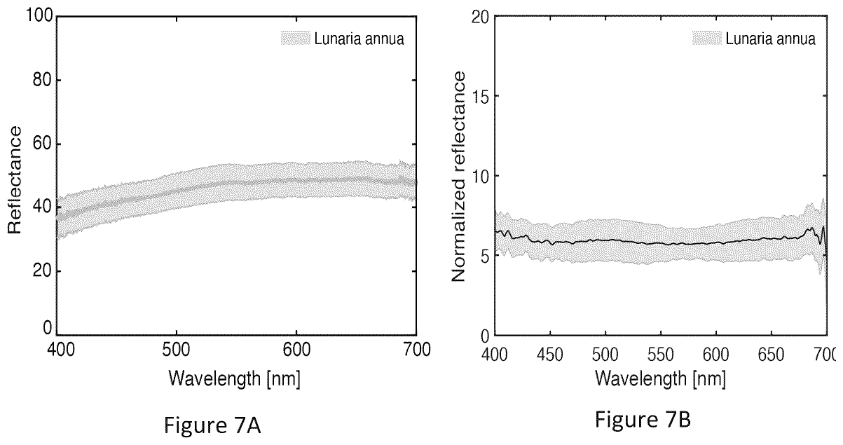

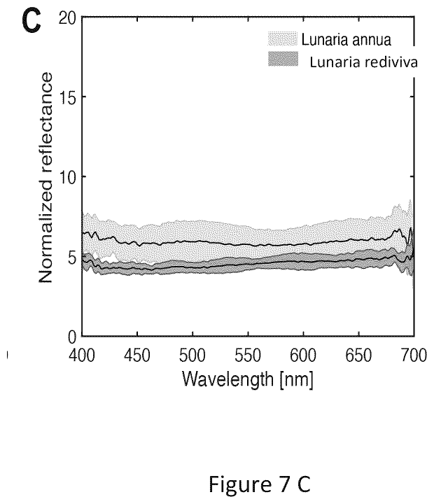

As can be seen in FIG. 6 and FIG. 7, the microscopic properties and reflectance of L. rediviva are comparable to those of L. annua. The L. rediviva septa also have morphology with a parallel orientation of cell fibrils demonstrating thin-film interference colors.

Methods of Forming Visible and/or UV-Blocking Composition

A reflective composition that blocks or attenuates UV and/or visible light transmission can be fabricated from dried Lunaria septa according to the following general steps: cooling, fragmenting, fractionating. The dried Lunaria septa can further be combined with a carrier.

Initially, the Lunaria can be harvested at a time when the seedpods are silvery-white and possess a broadband surface reflectance that is at least 40% of a normalized maximum for each wavelength between 400 nm and 700 nm. Alternatively, the Lunaria can be harvested when the silvery-white seedpods have a broadband reflectance that is at least 50%, at least 60%, at least 70%, at least 80%, or at least 90% of a normalized maximum for each wavelength between 400 nm and 700 nm.

In a first step, dried Lunaria septa can be isolated from the seedpod by any suitable means, such as mechanical, automated, or manual means. The dried Lunaria septa can be separated from the valves, seeds, replum, siliques, and any other residual components.

Once the dried Lunaria septa are isolated, the septa are coarsely ground or otherwise fragmented to form flakes or thin macroscopic particles of a few millimeters in length and width. In one example, the dried Lunaria septa can be fragmented by grinding using an electric grinder operating between 50 rpm and 20,000 rpm.

Under cryogenic conditions, the macroparticles can be further fragmented to form microparticles. Cooling to low temperatures causes the septa to become brittle and to fragment into flakes that maintain the microscopic structure of the septa.

The macroparticles can be cooled to a temperature below the freezing point of water for fragmenting. In one example, the macroparticles can be immersed in liquid nitrogen for at least 3 minutes to cool them to −196° C. Alternatively, the dried Lunaria septa can be cooled to a low temperature, for example, the temperature can be below −10° C., below −50° C., below −100° C., or below −150° C. by submerging the septa in a thermal bath.

The macroparticles can be finely ground to microparticles using a mill. In an example, the mill is a ball mill. In another example, the mill is a loot mill. In another example, the mill is a high-speed analytical mill with cooling. In one example, the septa can be fragmented using a ball mill operating at a speed between 15 rpm and 40,000 rpm while maintaining the temperature of the microparticles during grinding. In one example, the temperature can be maintained between 25° C. and −200° C. during the grinding. In another example, the temperature can be maintained between −15° C. and −200° C. during the grinding. In another example, the temperature can be maintained between −70° C. and −200° C. during the grinding. Alternatively, fragmenting can be accomplished by any mechanical means that apply shear forces, impact, or cavitation to form microparticles from the dried Lunaria septa. Additionally, and alternatively, the fragmenting can be accomplished using chemical means, for example oxidative reactions or enzymatic activity (e.g. lignase).

### Structure and Optical Properties of Microparticles

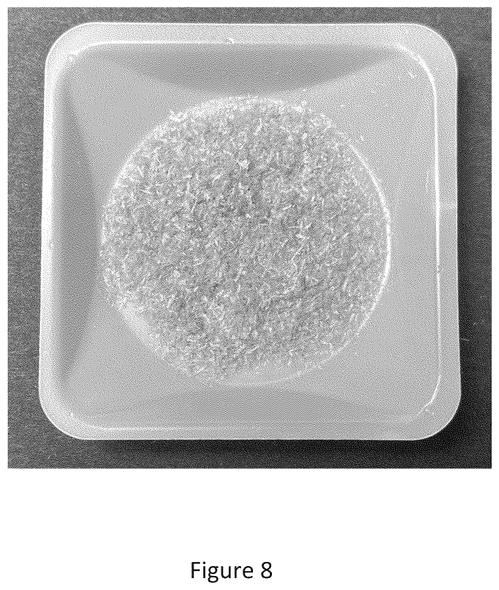

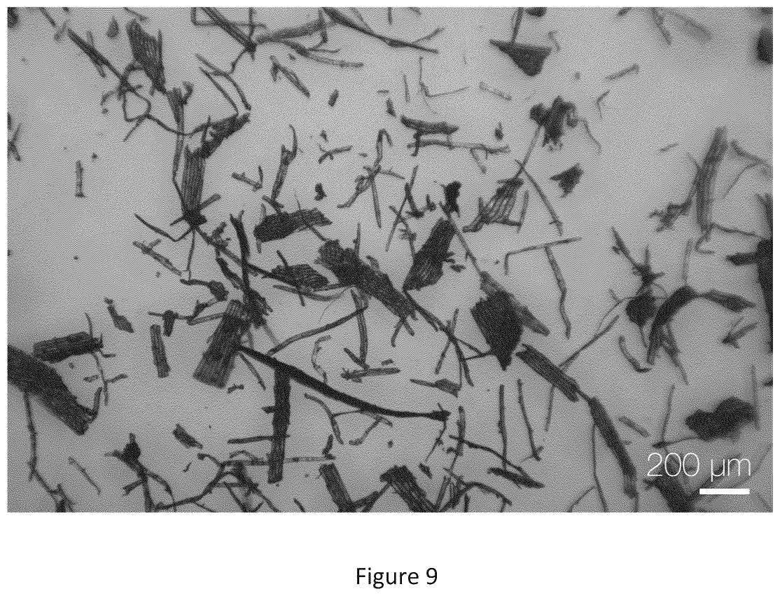

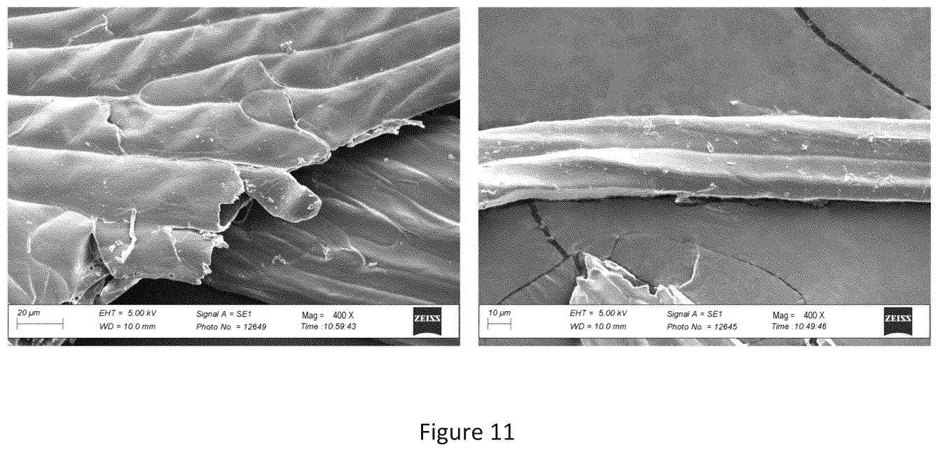

In one embodiment, the microparticles are carefully processed to maintain the microscopic structure and optical properties of the whole septa as shown in FIGS. 8-14. The microparticles obtained from the grinding of the Lunaria annua septa include bilayer assemblies of cellulose tubes, organized as hollow-core cylindrical cells. [1,2] The microparticles produce a glitter effect as can be seen in FIG. 8. As can be seen in FIG. 9, the microparticles can be characterized as thin fibers or flakes having uneven edges. The flake microparticles can include multiple parallel fibers as found in a domain. (FIGS. 9-11) A first distance between opposite sides of the microparticles can be between about 20 μm and 200 μm. A second distance between opposite sides of the microparticles can be between 1000 μm and 200 μm. In another case, the first distance can be between about 15 μm and 160 μm and the second distance can be between 50 μm and 380 μm. In one example, the microparticles can have an aspect ratio (such as first distance:second distance) of between 1:1 and 1:100. The thickness of the microparticles can be about 18 μm, based on the bilayer thickness measured in the cross-sectional view (FIG. 3A).

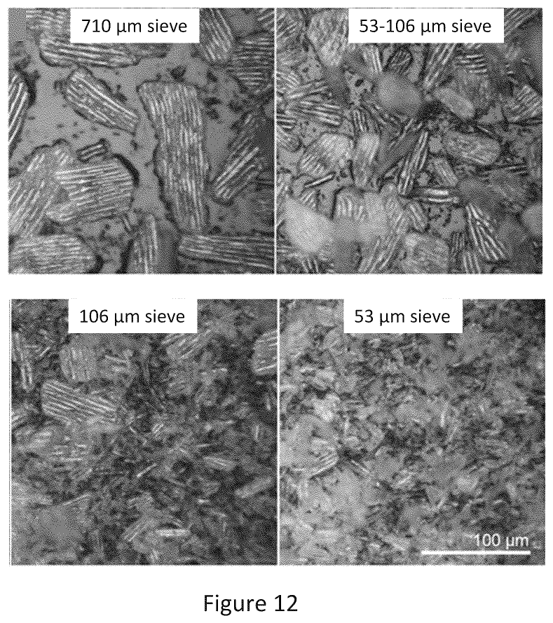

Fractionation can include separation of flakes, or microparticles having multiple adjacent fibers from microparticles having single fibers. The microparticles can be fractionated or sorted by size by any suitable means such as sieving, centrifugation, or sedimentation. In one example, as shown in FIG. 12, sieves with various mesh sizes such as 710 μm, 106 μm, and 53 μm can be used to sort the microparticles.

In FIG. 12, the panels labeled 710 μm, 106 μm, and 53 μm show the microparticles that went through the 710 μm, 106 μm, and 53 μm sieve, respectively. The 53-106 μm fraction consists of microparticles that went through the 106 μm sieve but not the 53 μm sieve. The fractions obtained using smaller mesh sizes show fewer microparticles that retained the thin-film interference colors, along with particles that lost that characteristic coloration. In this example, sieving allows size-sorting of the microparticles into fractions with microparticles having average width that ranges from (120±46) μm to (28±13) μm and length that ranges from (266±120) μm to (72±23) μm for mesh sizes of 710 μm and 53 μm, respectively (See Table 1).

TABLE 1
Size distribution of the microparticles width and length
after the sieving process. For each sieve mesh size,
the reported values are the average (avg) ± standard
deviation (st. dev.) for n measured microparticles.

| | Width | Length | |
|---|---|---|---|
| sieve mesh size | avg ± st. dev. | avg ± st. dev. | |
| [μm] | [μm] | [μm] | n |
| 710 | 120.3 ± 45.9 | 265.8 ± 119.7 | 96 |
| 106 | 45.9 ± 24.5 | 102.2 ± 46.9 | 100 |
| 53-106 | 77.1 ± 22.6 | 160.8 ± 56.8 | 100 |
| 53 | 28.4 ± 13.2 | 72.1 ± 22.8 | 100 |

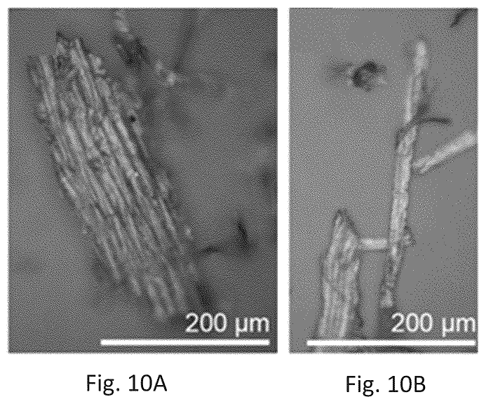

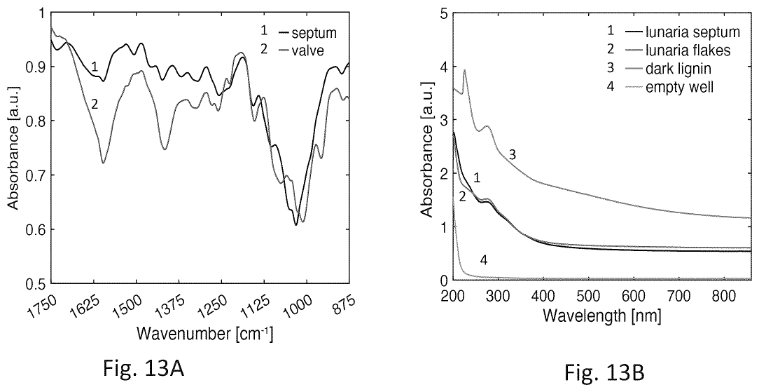

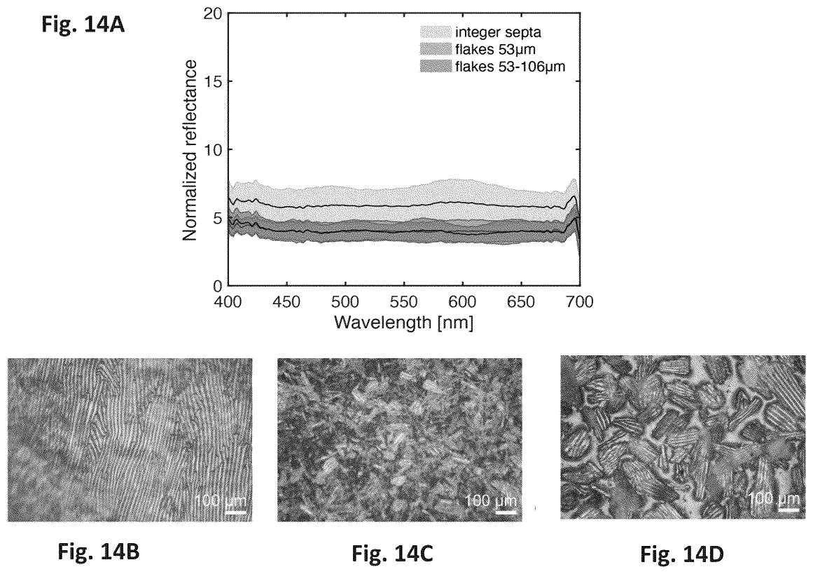

The glitter effect is observed following the grinding process, as can be seen in FIG. 8. The thin-film interference colors in individual cells and fibers are observed at higher magnification of the microparticles. (FIG. 10A) Unexpectedly, the careful generation of these smaller particles correlates with a shift in coloration from silvery-white to light gold due to changes in the lignin structure during processing. The optical properties of microparticles can be more specifically compared to the whole Lunaria septa (FIGS. 13-14). As can be seen in FIG. 13A, the FTIR spectral fingerprints at ~1596 cm⁻¹ and 1506 cm⁻¹ can be attributed to lignin compounds. FIG. 13 shows that the absorbance of Lunaria septa and Lunaria microparticles are comparable, while lower than the absorbance of a lignin control sample. The shift in coloration from silvery-white to light gold, corresponding to increased visible reflectance and UV absorbance, is highly unexpected as lignin is usually very dark due to the presence of chromophore inclusions.

Similarly, the normalized broadband reflectance across the visible range typical of the L. annua septa is similar for microparticles in the 53-106 μm fraction and 53 μm fraction as can be seen in the overlapping regions in FIG. 14A. The normalized broadband reflectance is somewhat lower for microparticles (~4%) as compared to the whole septa (~6%). FIGS. 14B-D show the structural differences between the whole septum with multidomain structure (FIG. 14B) and the flake microparticle fractions of 53-106 μm and 53 μm (FIGS. 14C and D) each having smaller domain representations. Thus, the processing of the septa results in fabrication of microscale highly reflective cellulose microparticles that can be used as UV and visible light filters.

### Carrier

After fractionation, the microparticles can be included in a composition. The composition of the present disclosure can be formulated into a variety of product forms including creams, lotions, mousses, gels, oils, and sprays. These product forms can be used for applications, including, but not limited to, hand and body lotions, facial creams, facial moisturizers. These product forms can be used for cosmetics such as make-up, foundation, lipstick, and the like. The light-scattering or glitter effect of the microparticles can imbue the cosmetic with an appealing aesthetic. Any additional components required to formulate such products vary with product type and can be routinely chosen by one skilled in the art. In some cases, the composition can be formulated into lotions, oils, hair care, lip, and other skincare products while maintaining sun-protective properties.

The composition can include a carrier. The carrier can be chosen according to the desired application. The carrier, or vehicle, suitable for application to human skin. As used herein a “carrier suitable for application to human skin,” means that the carrier and its components are suitable for use in contact with human skin without undue toxicity, incompatibility, instability, allergic response, and the like within the scope of sound medical or formulator's judgment. Such carriers are well known to one of ordinary skill in the art, and can include one or more compatible liquid or solid filler diluents or vehicles which are suitable for application to human skin. The carrier may comprise one or more active or inactive materials, including but not limited to, optional components described below.

The carrier can optionally be an emulsion. For example, suitable emulsions include oil-in-water, water-in-oil, water-in-oil-in-water, oil-in-water-in-oil, and oil-in-water-in-silicone emulsions.

The composition can be formulated as an aerosol and applied to the skin as a spray-on product, in which case a propellant can be added to the composition. Examples of suitable propellants include butane, isobutane, propane, isopentane, and chlorofluorinated lower molecular weight hydrocarbons.

The carrier can include one or more optional components including emulsifiers, emollients, artificial tanning agents, humectants, moisturizers, skin conditioners, UVA filters, UVB filters or thickening agent.

The carrier can include emulsifiers. Emulsifiers generally serve to reduce the in interfacial tension between phases and improve the formulation and stability of an emulsion. Suitable types of emulsifier include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps and mixtures thereof.

The carrier can optionally include an emollient. The emollient can include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

The carrier can optionally include one or more artificial tanning agents. Suitable tanning agents include dihydroxyacetone, tyrosine, tyrosine esters and phopho-pho-DOPA.

The carrier can optionally include one or more humectants, moisturizers, or skin conditioners. These materials include, but are not limited to, guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine; and mixtures thereof. The composition can in some cases be formulated as a gel-based formulation, which can optionally include hyaluronic acid, liquid shea butter, cyclopentasiloxane, dimethicone, dimethicone crosspolymer, trisiloxane, silica, dimethicone/vinyl dimethicone crosspolymer, ethylhexyl salicylate, retinyl palmitate, tocopheryl acetate, or the like.

The carrier can optionally include one or more thickening agents. Examples of thickening agents include carboxylic acid polymers, crosslinked polyacrylate polymers, polyacrylamide polymers, polysaccharides, and gums.

The carrier can optionally include one or more of a UV filter such as oxybenzone, octyl methoxycinnamate, homosalate, octisalate, octocrylene, meradimate, avobenzone, zinc oxide, or titanium dioxide. In another example, carrier can optionally include one or more of a naturally derived oil, such as raspberry oil, carrot oil, wheat germ oil, walnut oil, or buriti oil. In an example, carrier can optionally include one or more of propolis, shea butter, alder buckthorn components, or aloe vera. In a few specific cases, the composition can include particles suspended in plant-based oils, including but not limited to, Vitis Vinifera (Grape) Seed Extract, Cola Acuminata (Kola) Seed Extract, Camellia Oleifera Leaf Extract, Helianthus Annuus (Sunflower) Seed Oil, olive derived squalene oil, Ricinus Communis (Castor) Seed Oil, Butyrospermum Parkii (Shea) Oil, or the like. In some cases, the compositions can include plant extracts, such as prickly pear extract, watermelon extract, or the like.

The compositions of the present disclosure may contain a variety of optional ingredients. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, opacifying agents, pH adjusters, reducing agents, sequestrants, skin bleaching agents (or lightening agents) (e.g., hydroquinone, kojic acid, ascorbic acid, magnesium ascorbyl phosphate, ascorbyl glucosamine), skin-conditioning agents (humectants, including miscellaneous and occlusive), skin soothing and/or healing agents (e.g., panthenol and derivatives (e.g., ethyl panthenol), pantothenic acid and its derivatives, allantoin, bisabolol, and dipotassium glycyrrhizinate), skin treating agents including agents for preventing, retarding, arresting, and/or reversing skin wrinkles (e.g., alpha-hydroxy acids such as lactic acid and glycolic acid and beta-hydroxy acids such as salicylic acid), thickeners, and vitamins and derivatives thereof (e.g. tocopherol, tocopherol acetate, retinoic acid, retinol, retinoids, retinyl palmitate, niacin, niacinamide, and the like).

In one example, the Lunaria microparticles can be combined a sunscreen base, where the sunscreen base can include a thickening agent, an emulsifier, and a sensory additive. In another example, the carrier can be a skin cream such as an aloe cream.

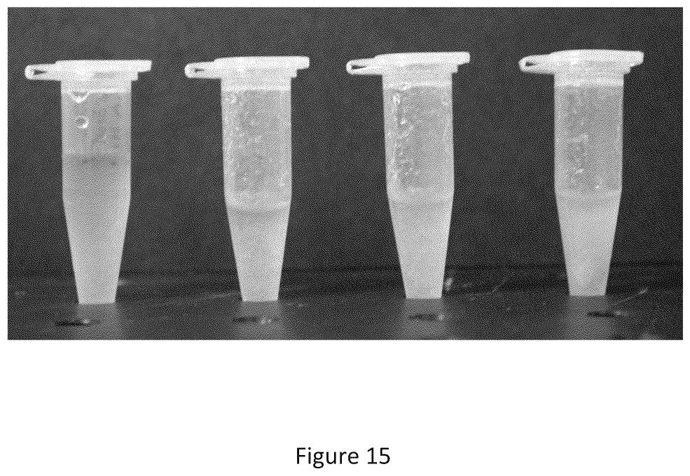

FIG. 15 (See Example 1) shows mixtures of microparticles in 0.3 wt %, 0.6 wt %, or 1.2 wt % with water-based aloe cream. Fractionated microparticles of any size grouping can be mixed with creams in a weight percentage between 0.1 wt % and 5 wt %. For example, microparticles can be combined with a carrier in loadings of 0.1 wt %, 0.2 wt %, 0.3 wt %, 0.4 wt %, 0.5 wt %, 0.6 wt %, 0.7 wt %, 0.8 wt %, 1.0 wt %, 1.1 wt %, 1.2 wt %, 1.3 wt %, 1.4 wt %, 1.5 wt % 1.6 wt %, 1.7 wt %, 1.8 wt %, 1.9 wt %, 2.0 wt %, 2.1 wt %, 2.2 wt %, 2.3 wt %, 2.4 wt %, 2.5 wt % 2.6 wt %, 2.7 wt %, 2.8 wt %, 2.9 wt %, 3.0 wt %, 3.1 wt %, 3.2 wt %, 3.3 wt %, 3.4 wt %, 3.5 wt % 3.6 wt %, 3.7 wt %, 3.8 wt %, 3.9 wt %, 4.0 wt %, 4.1 wt %, 4.2 wt %, 4.3 wt %, 4.4 wt %, 4.5 wt % 4.6 wt %, 4.7 wt %, 4.8 wt %, 4.9 wt %, or 5 wt %. For example, microparticles can be combined with a carrier in loadings of between 0.2 wt % and 0.3 wt %, between 0.4 wt % and 0.5 wt %, between 0.8 wt % and 1.2 wt %, between 1.5 wt % and 1.8 wt %, or between 3 wt % and 5 wt %. In yet another example, microparticles can be combined with a carrier in loadings of 0.24 wt %, 0.45 wt %, 1 wt %, 1.67 wt %, or 5 wt %.

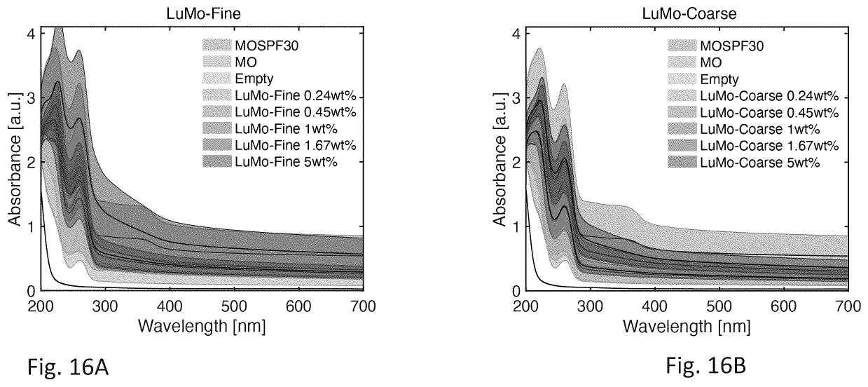

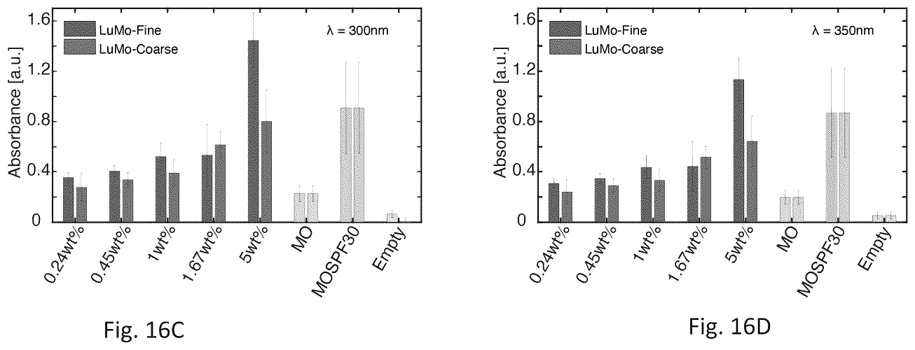

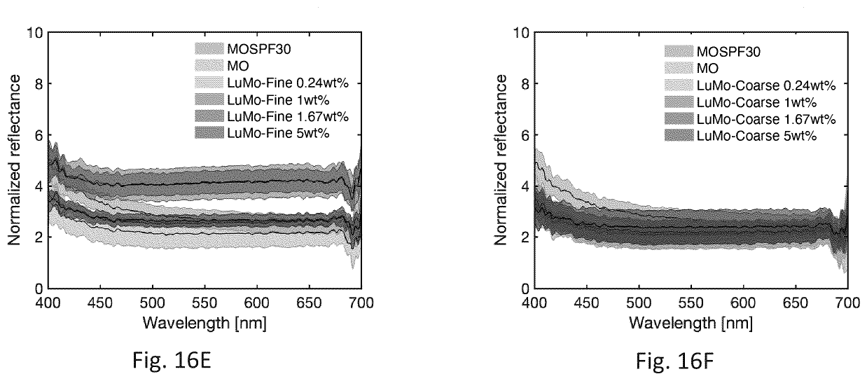

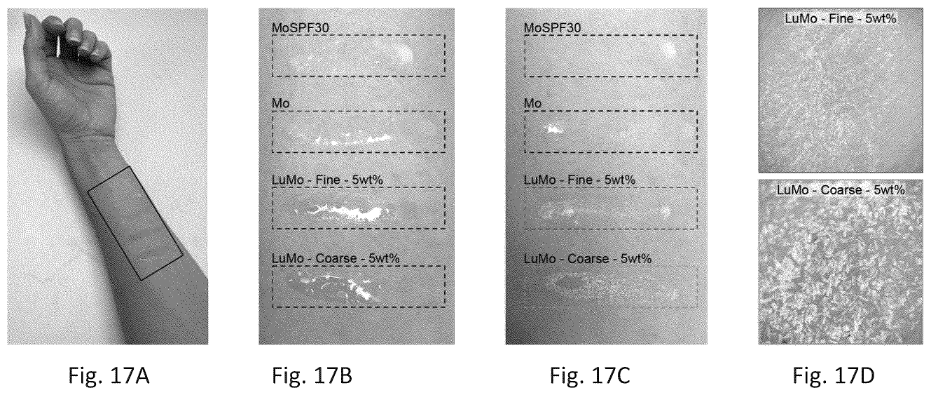

Microparticles of dried Lunaria septa with a carrier such as a cream for example a moisturizer or a sunscreen base, results in increased absorbance over the carrier alone. (Example 2, FIG. 16) The wt % loading of microparticles in the carrier can affect the final cream's light absorbance properties, especially in the UV range (λ=200-400 nm). (FIG. 16A-D). Increased loading or higher wt % of microparticles results in increased absorbance and increased reflectance. In this example, loading of ~5 wt % exhibited comparable absorbance properties to a commercial moisturizer with a Sun Protection Factor (MOSPF).

The size of the microparticles in the cream has some affect the improvement in the absorbance. In Example 2 and FIG. 16 the fine microparticles (53 μm) and the coarse microparticles (53-106 μm) both demonstrate increased absorbance over the carrier in the UV range (λ=200-400 nm).

Microparticles of dried Lunaria septa can be mixed with a cream such as a moisturizer or a sunscreen resulting in increased reflectance. (Example 2, FIG. 16 E-F) The wt % loading of microparticles in the cream can improve the reflectance especially in the visible range (λ=400-700 nm). In Example 2, creams with fine microparticles (53 μm) and coarse microparticles (53-106 um) both exhibit normalized reflectance comparable to or higher than the reflectance of the carriers (FIG. 16E-F).

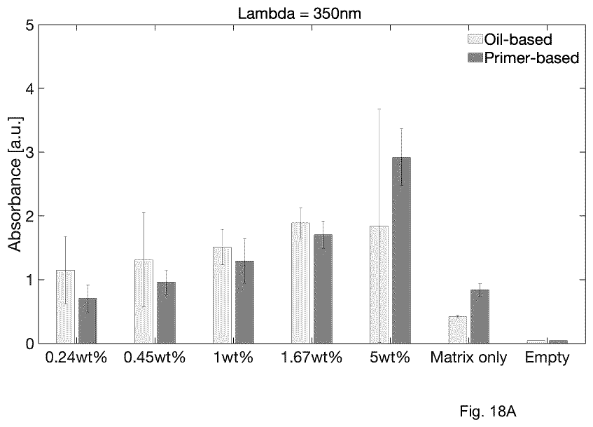

The optical appearance of the Lunaria-containing creams on skin can be used to determine the feasibility of the use of Lunaria creams as component in commercial sunscreens and skincare products (Example 3, FIG. 17). A visually glittery appearance can correspond to an improved reflectance of visible or UV light. For example, applying the microparticle cream to human skin can provide a broadband surface reflectance that is at least 40%, at least 50%, at least 60%, at least 70%, at least 80%, or at least 90% of light in the UV range, the visible range, or both. In Example 3, the optical appearance of the Lunaria creams on skin demonstrates the practicability of the Lunaria creams as sunscreen components (FIG. 17). The wet Lunaria creams at high loadings (5 wt %) have a golden appearance both for the fine and the coarse microparticles (FIG. 17B); upon drying the Lunaria microparticles remain on the surface of the skin (FIGS. 17C and D). These microparticles can be removed from the skin surface by flushing with water.

### Applications

The application of the dried Lunaria septa microparticles are not limited to skin care as described herein. According to an aspect of the disclosure herein, the UV-blocking and/or visible light-blocking composition can be a powder or a dried film. In another embodiment, the UV-blocking and/or visible light-blocking composition can include incorporating the microparticles into a carrier such that the UV-blocking composition can be used to manufacture packaging for light-sensitive materials. In a further embodiment, the UV-blocking and/or visible light-blocking composition can include a carrier that allows the UV-blocking and/or visible light-blocking composition to be used as a cosmetic. In a still further embodiment, the UV-blocking and/or visible light-blocking composition can include a carrier that makes the UV-blocking and/or visible light-blocking composition suitable as a paint. The light weight of the particles themselves combined with the UV-blocking and/or visible light-blocking composition could also be used in textile manufacturing to produce sun-protecting fabrics.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, any of the features or functions of any of the embodiments disclosed herein may be incorporated into any of the other embodiments disclosed herein.

### EXAMPLES

### Example 1

A 25 wt % water-based aloe cream is used to suspend the Lunaria flakes in 0.3 wt %, 0.6 wt %, or 1.2 wt % respectively. (FIG. 15) Further dilutions of aloe cream in water (such as 15 wt % and 10 wt %) induce sedimentation of the flakes due to the lower cream viscosity. Upon shaking the suspension gets easily redispersed. The appearance of the microparticles is golden yellow and shimmery. The yellow appearance is due to the lignin grinding and to the decrease of the additive color mixing principle. The 25 wt % water-based aloe cream is 25% by weight aloe cream in water.

### Example 2

Microparticles of dried Lunaria septa were mixed with 15 wt % of either a moisturizer composition (“MO”) or a moisturizer composition containing a combination of commercially-available organic and mineral compounds that interact with and absorb the energy from UV radiation (Homosalate 10%, Meradimate 5%, Octinoxate 5%, Octocrylene 2% and Zinc Oxide 6.3%) (“MOSPF”) at 0.24 wt %, 0.45 wt %, 1 wt %, 1.67 wt %, and 5 wt % microparticle loadings to obtain a fine and a coarse series of creams: “LuMo Fine” made with particles of the 53 μm fraction and “LuMo Coarse” made with particles of the 53-106 μm fraction. Increasing loading of the microparticles resulted in increased absorbance especially in the λ=200-400 nm range for both the “LuMo Fine” and the “LuMo Coarse” (FIG. 16A-D). By evaluating the UV-absorbance at λ=300 nm (FIG. 16C) and at X=350 nm (FIG. 16D), the increase of the absorbance as a function of the loading of the microparticles can be clearly seen. Both for the fine and coarse microparticles, loading of ~5 wt % exhibited comparable absorbance properties to the commercial sunscreen MOSPF, which claims to provide SFP 30 sun protection. In terms of normalized reflectance evaluated over the visible range (X=400-700 nm) both series of creams exhibit comparable, if not higher, normalized reflectance compared to the commercial moisturizing lotion and to the sunscreen cream (FIG. 16E-F).

### Example 3

Samples of the “LuMo Fine” and the “LuMo Coarse” 5 wt % creams were applied to a human volunteer's arm and allowed to dry for visual inspection. (FIG. 17) (A) The black rectangle highlights the region of the arm where the samples were applied. (B) Macroscopic pictures of the cream swatches just applied on the skin (B) and after drying (C): from top to bottom, swatch of the commercially available sunscreen cream (MOSPF30), the moisturizing cream (MO), the fine Lunaria-based cream at 5 wt %, and the coarse Lunaria-based cream at 5 wt %. (D) The wet Lunaria creams at high loadings (5 wt %) have a golden appearance both for the fine and the coarse microparticles (FIG. 17B); upon drying the Lunaria microparticles remain reflective on the surface of the skin (FIGS. 17C and D).

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Example 4

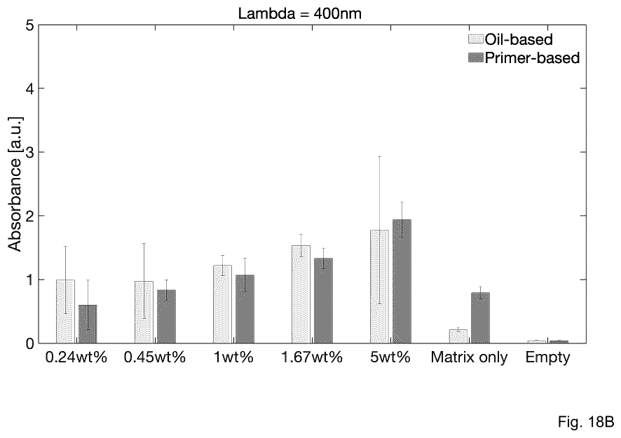

Spectral analysis of various Lunaria annua solutions was performed with different carriers, including grapeseed oil and primer. For spectroscopic analysis, these samples were diluted in isopropyl alcohol (IPA) to reduce viscosity, enhance mixing, and reduce background UV absorbance of the carrier. Fine particles of Lunaria annua at various wt % were prepared using oil and primer as matrix (e.g., carrier). Dilutions were prepared to have Lunaria annua at 0.24 wt %, 0.45 wt %, lwt %, 1.67 wt %, and 5 wt %. As control, 20 wt % oil in IPA and 22 wt % primer in IPA were used (matrix only) as well as the empty well (empty). Referring to FIG. 18A, the absorbance [a.u.] at a wavelength of 350 nm is depicted for the Lunaria samples and FIG. 18B depicts the absorbance at 400 nm. For both wavelengths, there is an increase in absorbance with increasing Lunaria particle concentration.

A composition was also prepared by suspending the same particle compositions in a serum formulation, but the serum formulation was not capable of adequate dilution for spectroscopic observations. It is expected that the performance in serum formulations will follow similar trends as those observed in the other carriers.

### REFERENCES

[1] Urban, K. et al, JAAD International, vol. 2, p. 98-108, 2021; https://doi.org/10.1016/j.jdin.2020.10.013.
[2]Sánchez-Quiles, D., et al., Environ. Sci. Technol. 2014, 48, 16, 9037-9042.
[3]C. Leins, P. Fligge, K. Erbar, Plant Biol. 2018, 20, 238.
[4]G. Guidetti, H. Sun, B. Marelli, F. G. Omenetto, Sci. Adv. 2020, 6, eaba8966.

1.-2. (canceled)

3. The method of claim 31, wherein the Lunaria septa are from Lunaria plants harvested at a time when their seedpods possess a broadband surface reflectance that is at least 40%, at least 50%, at least 60%, at least 70%, at least 80%, or at least 90% of a normalized maximum for each wavelength between 400 nm and 700 nm, wherein the broadband surface reflectance is normalized to a white diffuser.

4. The method of claim 3, wherein the seedpods have a white or yellow appearance.

5.-10. (canceled)

11. The method of claim 31, wherein the cellulose microparticles have an aspect ratio of between 1:1 and 1:100.

12. The method of claim 31, wherein the cellulose microparticles are fractionated using at least one of at least one sieve or centrifugation.

13. (canceled)

14. The method of claim 12, wherein fractionating the cellulose microparticles comprises separating microparticles having multiple adjacent fibers from microparticles having single fibers.

15. The method of claim 31, further comprising reducing the Lunaria septa to macroparticles before fragmenting the Lunaria septa to microparticles while maintaining a temperature of the Lunaria septa at or below −15° C.

16. The method of claim 15, wherein the Lunaria septa are reduced to macroparticles by a grinder with a grinding component rotating between 500 rpm and 20,000 rpm.

17. The method of claim 31, wherein the cellulose microparticles are characterized by reflectance between 400 nm and 700 nm.

18. The method of claim 31, wherein the cellulose microparticles are characterized by absorbance between 200 nm and 400 nm.

19. The method of claim 31, wherein the at least one carrier is a sunscreen base and comprises at least one of the following: a UVA filter, a UVB filter, an emollient, a moisturizer, a thickening agent, an emulsifier, or a sensory additive.

20. The method of claim 31, wherein the at least one carrier is a packaging material and comprises at least one of the following: a thermoplastic material, a polymer, monomers, or an adhesive.

21.-22. (canceled)

23. The visually glittery composition of claim 32, wherein the visually glittery composition is a cream.

24. The visually glittery composition of claim 32, wherein the visually glittery composition is a powder.

25. The visually glittery composition of claim 32, wherein the visually glittery composition is a dried film.

26.-28. (canceled)

29. The method of claim 31, wherein the Lunaria is Lunaria annua, Lunaria rediviva, or Lunaria telekiana.

30. (canceled)

31. A method of using cellulose microparticles for visible light protection and/or UV light protection, the method comprising:
applying a formulation to skin, the formulation comprising an at least 1 wt % particle concentration of cellulose microparticles from Lunaria septa in at least one carrier,
wherein the cellulose microparticles are between 10 μm and 100 μm in diameter, wherein the cellulose microparticles show at least 50% of a normalized maximum for each wavelength between 400 nm and 700 nm, and wherein reflectance is normalized to a white diffuser.

32. A visually glittery composition comprising: a particle comprising greater than 50% cellulose, wherein the particle comprises an array of parallel-arranged cellulose tubes, each tube having a cross-sectional diameter between 10 μm and 30 μm, and wherein the particle shows at least 50% of a normalized maximum for each wavelength between 400 nm and 700 nm, wherein reflectance is normalized to a white diffuser.

33. The visually glittery composition of claim 32, wherein the particle possesses a broadband surface reflectance that is at least 40%, at least 50%, at least 60%, at least 70%, at least 80%, or at least 90% of a normalized maximum for each wavelength between 400 nm and 700 nm, wherein the reflectance is normalized to a white diffuser.

34. The visually glittery composition of claim 32, the visually glittery composition further comprising a carrier suitable for use on human skin, wherein the carrier comprises at least one of the following components: a UVA filter, a UVB filter, an emollient, a moisturizer, or a thickening agent.

35. The visually glittery composition of claim 32, wherein the particle imbues the visually glittery composition with light-scattering effects.
